SEQUENCE LISTING (75 sequences)

SEQ ID NO: 1 - protein, 21 aa, Herpes Simplex
ASLKMADPNRFRGKDLPVLDQ

SEQ ID NO: 2 - protein, 21 aa, Herpes Simplex
ASLKMADPNRFRGKDLPVLDR

SEQ ID NO: 3 - protein, 21 aa, Herpes Simplex
ASLKMADPNRFRGKDLPVLDP

SEQ ID NO: 4 - protein, 21 aa, Herpes Simplex
ASLKMADPNRFRGKDLPVPDQ

SEQ ID NO: 5 - protein, 21 aa, Herpes Simplex
ASLKMADPNRFRGKDLPVPDR

SEQ ID NO: 6 - protein, 21 aa, Herpes Simplex
ASLKMADPNRFRGKDLPVPDP

SEQ ID NO: 7 - protein, 21 aa, Herpes Simplex
ASLKMADPNRFRGKNLPVLDQ

SEQ ID NO: 8 - protein, 21 aa, Herpes Simplex
ASLKMADPNRFRGKNLPVLDR

SEQ ID NO: 9 - protein, 21 aa, Herpes Simplex
ASLKMADPNRFRGKNLPVLDP

SEQ ID NO: 10 - protein, 21 aa, Herpes Simplex
ASLKMADPNRFRGKNLPVPDQ

SEQ ID NO: 11 - protein, 21 aa, Herpes Simplex
ASLKMADPNRFRGKNLPVPDR

SEQ ID NO: 12 - protein, 21 aa, Herpes Simplex
ASLKMADPNRFRGKNLPVPDP

SEQ ID NO: 13 - protein, 21 aa, Herpes Simplex
PSLKMADPNRFRGKDLPVLDQ

SEQ ID NO: 14 - protein, 21 aa, Herpes Simplex
PSLKMADPNRFRGKDLPVLDR

SEQ ID NO: 15 - protein, 21 aa, Herpes Simplex
PSLKMADPNRFRGKDLPVLDP

SEQ ID NO: 16 - protein, 21 aa, Herpes Simplex
PSLKMADPNRFRGKDLPVPDQ

SEQ ID NO: 17 - protein, 21 aa, Herpes Simplex
PSLKMADPNRFRGKDLPVPDR

SEQ ID NO: 18 - protein, 21 aa, Herpes Simplex
PSLKMADPNRFRGKDLPVPDP

SEQ ID NO: 19 - protein, 21 aa, Herpes Simplex
PSLKMADPNRFRGKNLPVLDQ

SEQ ID NO: 20 - protein, 21 aa, Herpes Simplex
PSLKMADPNRFRGKNLPVLDR

SEQ ID NO: 21 - protein, 21 aa, Herpes Simplex
PSLKMADPNRFRGKNLPVLDP

SEQ ID NO: 22 - protein, 21 aa, Herpes Simplex
PSLKMADPNRFRGKNLPVPDQ

SEQ ID NO: 23 - protein, 21 aa, Herpes Simplex
PSLKMADPNRFRGKNLPVPDR

SEQ ID NO: 24 - protein, 21 aa, Herpes Simplex
PSLKMADPNRFRGKNLPVPDP

SEQ ID NO: 25 - protein, 33 aa, Herpes Simplex
KYALADASLKMADPNRFRGKDLPVLDQLTDPPG

SEQ ID NO: 26 - protein, 33 aa, Herpes Simplex
KYALADASLKMADPNRFRGKDLPVLDRLTDPPG

SEQ ID NO: 27 - protein, 33 aa, Herpes Simplex
KYALADASLKMADPNRFRGKDLPVLDPLTDPPG

SEQ ID NO: 28 - protein, 33 aa, Herpes Simplex
KYALADASLKMADPNRFRGKDLPVPDQLTDPPG

SEQ ID NO: 29 - protein, 33 aa, Herpes Simplex
KYALADASLKMADPNRFRGKDLPVPDRLTDPPG

SEQ ID NO: 30 - protein, 33 aa, Herpes Simplex
KYALADASLKMADPNRFRGKDLPVPDPLTDPPG

SEQ ID NO: 31 - protein, 33 aa, Herpes Simplex
KYALADASLKMADPNRFRGKNLPVLDQLTDPPG

SEQ ID NO: 32 - protein, 33 aa, Herpes Simplex
KYALADASLKMADPNRFRGKNLPVLDRLTDPPG

SEQ ID NO: 33 - protein, 33 aa, Herpes Simplex
KYALADASLKMADPNRFRGKNLPVLDPLTDPPG

SEQ ID NO: 34 - protein, 33 aa, Herpes Simplex
KYALADASLKMADPNRFRGKNLPVPDQLTDPPG

SEQ ID NO: 35 - protein, 33 aa, Herpes Simplex
KYALADASLKMADPNRFRGKNLPVPDRLTDPPG

SEQ ID NO: 36 - protein, 33 aa, Herpes Simplex
KYALADASLKMADPNRFRGKNLPVPDPLTDPPG

SEQ ID NO: 37 - protein, 33 aa, Herpes Simplex
KYALADPSLKMADPNRFRGKDLPVLDQLTDPPG

SEQ ID NO: 38 - protein, 33 aa, Herpes Simplex
KYALADPSLKMADPNRFRGKDLPVLDRLTDPPG

SEQ ID NO: 39 - protein, 33 aa, Herpes Simplex
KYALADPSLKMADPNRFRGKDLPVLDPLTDPPG

SEQ ID NO: 40 - protein, 33 aa, Herpes Simplex
KYALADPSLKMADPNRFRGKDLPVPDQLTDPPG

SEQ ID NO: 41 - protein, 33 aa, Herpes Simplex
KYALADPSLKMADPNRFRGKDLPVPDRLTDPPG

SEQ ID NO: 42 - protein, 33 aa, Herpes Simplex
KYALADPSLKMADPNRFRGKDLPVPDPLTDPPG

SEQ ID NO: 43 - protein, 33 aa, Herpes Simplex
KYALADPSLKMADPNRFRGKNLPVLDQLTDPPG

SEQ ID NO: 44 - protein, 33 aa, Herpes Simplex
KYALADPSLKMADPNRFRGKNLPVLDRLTDPPG

SEQ ID NO: 45 - protein, 33 aa, Herpes Simplex
KYALADPSLKMADPNRFRGKNLPVLDPLTDPPG

SEQ ID NO: 46 - protein, 33 aa, Herpes Simplex
KYALADPSLKMADPNRFRGKNLPVPDQLTDPPG

SEQ ID NO: 47 - protein, 33 aa, Herpes Simplex
KYALADPSLKMADPNRFRGKNLPVPDRLTDPPG

SEQ ID NO: 48 - protein, 33 aa, Herpes Simplex
KYALADPSLKMADPNRFRGKNLPVPDPLTDPPG

SEQ ID NO: 49 - protein, 33 aa, Herpes Simplex
KYALVDASLKMADPNRFRGKDLPVLDQLTDPPG

SEQ ID NO: 50 - protein, 33 aa, Herpes Simplex
KYALVDASLKMADPNRFRGKDLPVLDRLTDPPG

SEQ ID NO: 51 - protein, 33 aa, Herpes Simplex
KYALVDASLKMADPNRFRGKDLPVLDPLTDPPG

SEQ ID NO: 52 - protein, 33 aa, Herpes Simplex
KYALVDASLKMADPNRFRGKDLPVPDQLTDPPG

SEQ ID NO: 53 - protein, 33 aa, Herpes Simplex
KYALVDASLKMADPNRFRGKDLPVPDRLTDPPG

SEQ ID NO: 54 - protein, 33 aa, Herpes Simplex
KYALVDASLKMADPNRFRGKDLPVPDPLTDPPG

SEQ ID NO: 55 - protein, 33 aa, Herpes Simplex
KYALVDASLKMADPNRFRGKNLPVLDQLTDPPG

SEQ ID NO: 56 - protein, 33 aa, Herpes Simplex
KYALVDASLKMADPNRFRGKNLPVLDRLTDPPG

SEQ ID NO: 57 - protein, 33 aa, Herpes Simplex
KYALVDASLKMADPNRFRGKNLPVLDPLTDPPG

SEQ ID NO: 58 - protein, 33 aa, Herpes Simplex
KYALVDASLKMADPNRFRGKNLPVPDQLTDPPG

SEQ ID NO: 59 - protein, 33 aa, Herpes Simplex
KYALVDASLKMADPNRFRGKNLPVPDRLTDPPG

SEQ ID NO: 60 - protein, 33 aa, Herpes Simplex
KYALVDASLKMADPNRFRGKNLPVPDPLTDPPG

SEQ ID NO: 61 - protein, 33 aa, Herpes Simplex
KYALVDPSLKMADPNRFRGKDLPVLDQLTDPPG

SEQ ID NO: 62 - protein, 33 aa, Herpes Simplex
KYALVDPSLKMADPNRFRGKDLPVLDRLTDPPG

SEQ ID NO: 63 - protein, 33 aa, Herpes Simplex
KYALVDPSLKMADPNRFRGKDLPVLDPLTDPPG

SEQ ID NO: 64 - protein, 33 aa, Herpes Simplex
KYALVDPSLKMADPNRFRGKDLPVPDQLTDPPG

SEQ ID NO: 65 - protein, 33 aa, Herpes Simplex
KYALVDPSLKMADPNRFRGKDLPVPDRLTDPPG

SEQ ID NO: 66 - protein, 33 aa, Herpes Simplex
KYALVDPSLKMADPNRFRGKDLPVPDPLTDPPG

SEQ ID NO: 67 - protein, 33 aa, Herpes Simplex
KYALVDPSLKMADPNRFRGKNLPVLDQLTDPPG

SEQ ID NO: 68 - protein, 33 aa, Herpes Simplex
KYALVDPSLKMADPNRFRGKNLPVLDRLTDPPG

SEQ ID NO: 69 - protein, 33 aa, Herpes Simplex
KYALVDPSLKMADPNRFRGKNLPVLDPLTDPPG

SEQ ID NO: 70 - protein, 33 aa, Herpes Simplex
KYALVDPSLKMADPNRFRGKNLPVPDQLTDPPG

SEQ ID NO: 71 - protein, 33 aa, Herpes Simplex
KYALVDPSLKMADPNRFRGKNLPVPDRLTDPPG

SEQ ID NO: 72 - protein, 33 aa, Herpes Simplex
KYALVDPSLKMADPNRFRGKNLPVPDPLTDPPG

SEQ ID NO: 73 - protein, 8 aa, Herpes Simplex
DQLTDPPG

SEQ ID NO: 74 - protein, 8 aa, Herpes Simplex
DRLTDPPG

SEQ ID NO: 75 - protein, 8 aa, Herpes Simplex
DPLTDPPG

(19) United States
(12) Patent Application Publication
(10) Pub. No.: US 2002/0090382 A1
(43) Pub. Date: Jul. 11, 2002

(54) Antiviral compounds and methods

(75) Inventor: Joseph C. Glorioso, Cheswick, PA (US)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(73) Assignee: University of Pittsburgh of the Commonwealth System of Higher Education, Pittsburgh, PA

(21) Appl. No.: 09/920,975

(22) Filed: Aug. 1, 2001

Related U.S. Application Data
(60) Provisional application No. 60/222,377, filed on Aug. 1, 2000.

(51) Int. Cl. A61K 39/12; C07K 14/01

(52) U.S. Cl. 424/204.1; 530/395

(57) ABSTRACT

The invention provides a polypeptide derived from the glycoprotein D (gD) of an HSV strain and to compositions including such polypeptides. The invention also provides prophylactic devices coated with such compositions. Using such reagents, the invention provides a method of reducing the probability of HSV or HIV infection of a cell and also reducing the probability of HSV or HIV transmission from an HSV⁺ or HIV⁺ individual to an HSV⁻ or HIV⁻ individual during physical contact. Furthermore, the invention provides a method to increase the likelihood that a prophylactic device will resist HSV or HIV infection of an individual.

### CROSS-REFERENCE TO RELATED APPLICATIONS

This application claims priority to United States Provisional Patent Application No. 60/222,377, filed on Aug. 1, 2000.

### STATEMENT REGARDING FEDERALLY SPONSORED RESEARCH AND DEVELOPMENT

This invention was made in part with Government support under Grant Numbers HL66949-01 and GM34534-18 from the United States National Institutes of Health. The United States Government may have certain rights to this invention.

### TECHNICAL FIELD OF THE INVENTION

This invention pertains to compounds and methods for reducing the likelihood of viral infection.

### BACKGROUND OF THE INVENTION

At present, no cure for infection with HSV strains (e.g., HSV-1 and HSV-2) or HIV is known, nor is there an effective vaccine against such viruses. Available technology to contain such infections and recurrent outbreaks involves the use of antiviral drugs that inhibit the viral DNA or RNA polymerases. One limitation to this form of treatment is that these drugs are generally only effective during the active stage of the viral life cycle. In addition, when administered at high doses these drugs also can affect the host-cell polymerases and thus can be toxic to patients. Another limitation is that although these drugs can lessen the symptoms of infection, they do not block the spread of the virus from an infected individual to an uninfected host.

Many vaccine approaches have been tried to block HSV and HIV infection; the most notable are described as being either inactivated or attenuated. The inactivated vaccines are produced by treating the viruses with chemical agents or by exposure to γ-rays so as to render them non-virulent. This type of virus produces mainly humoral immunity. Attenuated vaccines differ in that selection for a virulent organism takes place by growing a pathogen under adverse culture conditions or prolonged passage of a virulent human pathogen in different hosts. The benefit over the inactivated version is that both humoral and cell-mediated immunity are achieved. While such vaccines can mediate immunity in some animal models, there are drawbacks to their use in humans. For example, the attenuated vaccine can revert to virulent form, and thus initiate a partial or full-blown infection. While the inactivated vaccine cannot revert to its virulent form, multiple boosters typically are required to maintain an effective immunological response.

In light of the drawbacks attributed to attenuated and inactive viral vaccines, interest has risen for the production of novel vaccines that will have a prolonged effect on the host but will not carry the risk of reversion. Recombinant vector vaccines, for example, make it possible to introduce genes encoding antigens of the virulent pathogens into attenuated viruses. This allows for the attenuated virus to serve as a vector, replicating within the host while expressing the gene product of the pathogen. DNA vaccines, in which plasmid DNA encoding antigens of virulent pathogens is injected directly into the recipient, represents another novel approach to vaccination. In theory, such approaches aim to initiate a similar immune reaction as an attenuated vaccine but without the chance of infecting the recipient with the disease.

Another novel approach to vaccination is the use of synthetic polypeptides as vaccines. For example, such vaccines can consist of a number of amino acids derived from the desired pathogen, either alone or with an adjuvant, such as Freund's adjuvant, aluminum hydroxide, and aluminum potassium sulfate (alum). Given its role in mediating HSV infection, others have proposed using HSV glycoprotein D (gD) peptides and derivatives for blocking HSV infection or as protein-based vaccines. For example, U.S. Pat. Nos. 5,814,486 and 5,654,174 describe a peptide consisting of replacing amino acids 290-299 of gD with arg, lys, isoleu, and phen, as well as replacing amino acids 308-369 with five his residues. U.S. Pat. No. 5,851,533 describes a carboxy truncated form of gD for use as a vaccine. Furthermore, the '533 patent states that a vaccine which includes a mixture of gC and gD would be significantly more effective than either glycoprotein alone. U.S. Pat. No. 4,891,315 describes a method for the production of vaccines protective against HSV infection that comprises a variant gD 2 peptide. Finally, U.S. Pat. No. 4,709,011 describes a number of gD peptides consisting of 16 or 23 amino acid residues common to both gD-1 and gD-2, are cumulatively hydrophilic in nature, and specifically immunoreactive with a type common, monoclonal anti-gD antibody of Group VII classification. Despite such suggestions, approaches aimed at blocking HSV and HIV infection in humans or to immunize patients have failed, despite some efficacy demonstrated in animal studies. Therefore, a need exists for technology for protecting patients against HSV and HIV infection and also for an improved vaccine.

### BRIEF SUMMARY OF THE INVENTION

The invention provides a polypeptide derived from the glycoprotein D (gD) of an HSV strain and to compositions including such polypeptides. The invention also provides prophylactic devices coated with such compositions. Using such reagents, the invention provides a method of reducing the probability of HSV or HIV infection of a cell and also reducing the probability of transmission from an HSV⁺ or HIV⁺ individual to an HSV⁻ or HIV⁻ individual during physical contact. Furthermore, the invention provides a method to increase the likelihood that a prophylactic device will resist HSV or HIV infection of an individual. These and other advantages, as well as additional inventive features, will become apparent after reading the following detailed description, in conjunction with the accompanying drawings and sequence listing.

### BRIEF DESCRIPTION OF THE DRAWINGS

FIG. 1 graphically depicts the results of experiments concerning the effects of peptide blocking agents on subsequent HSV-1 infection of Vero cells.

FIG. 2 graphically depicts the results of experiments concerning the effects of peptide blocking agents on subsequent HSV-1 infection of HCO-HveA cells.

FIG. 3 graphically depicts the results of experiments concerning the effects of peptide blocking agents on subsequent HSV-1 infection of CHO-HveC cells.

FIG. 4 graphically depicts the binding affinity of gD peptides to HveA.

FIG. 5 graphically depicts the binding affinity of gD peptides to HveC.

### DETAILED DESCRIPTION OF THE INVENTION

The invention provides a polypeptide having an amino acid sequence derived from the amino-terminal domain of an HSV-1 or HSV-2 gD protein. In this regard, the sequences of the gD protein from many HSV strains are known (see, e.g., Izumi et al., J Exp. Med,. 172(2), 487-96 (1990), Lasky et al., DNA, 3(1):23-9 (1984Watson et al, Gene, 26(2-3), 307-12 (1983), Watson et al., Science, 218(4570), 381-84 (1982)), and any of these known proteins can serve as a source for the inventive polypeptide. The inventive polypeptide typically will comprise or consist essentially of from about 5 or about 10 or about 15 or about 20 amino acids to about 25 or about 30, or about 35 or about 40 or about 45 or even about 50 (preferably contiguous) amino acids from among the 55 amino-terminal amino acids of a gD protein. Preferably the inventive polypeptide includes at least a sequence of amino acids corresponding to amino acids 26-33 of the native gD sequence (e.g., SEQ ID NOs:73-75) or conservative substitutions thereof. While in many embodiments, the inventive polypeptide comprises no more than about 35 amino acids (e.g., from about 20 to about 30 amino acids), in some embodiments the inventive protein can comprise most of an HSV gD protein. In any event, the inventive polypeptide differs from a wild-type HSV gD protein at least one amino acid residue (e.g., the inventive protein comprises at least one point mutation relative to a wild-type HSV gD sequence).

An exemplary polypeptide of the instant invention can have a contiguous sequence of amino acids comprising or consisting essentially of those set forth as SEQ ID NOs:25-72 (which includes SEQ ID NOs:73-75); however, the inventive polypeptide is not limited to the exemplary sequences. For example, the inventive polypeptide typically can have an amino acid sequence at least about 75% homologous or identical to one of SEQ ID NOs:25-75 or conservative mutants thereof, preferably at least about 80% homologous or identical to one of SEQ ID NOs:25-75 or conservative mutants thereof (e.g., at least about 85% homologous or identical to one of SEQ ID NOs:25-75 or conservative mutants thereof). More preferably, the inventive polypeptide has an amino acid sequence at least about 90% homologous or identical to one of SEQ ID NOs:25-75 or conservative mutants thereof (such as at least about 95% homologous or identical to one of SEQ ID NOs:25-75 or conservative mutants thereof). Most preferably, the inventive polypeptide has an amino acid sequence at least about 97% homologous or identical to one of SEQ ID NOs:25-75 or conservative mutants thereof. Homology in this context means sequence similarity or identity, with identity being preferred. Identical in this context means identical amino acids at corresponding positions in the two sequences which are being compared. Homology in this context includes amino acids which are identical and those which are similar (functionally equivalent). This homology can be determined using standard techniques known in the art, such as the Best Fit sequence program described by Devereux, et al., Nucl. Acid Res., 12, 387-95 (1984), or the BLASTX program (Altschul, et al., J. Mol. Biol., 215, 403-10 (1990)) preferably using the default settings for either. In determining homology, the alignment can include the introduction of gaps in the sequences to be aligned. In addition, for sequences that contain either more or fewer amino acids than an optimum sequence, the percentage of homology can be determined based on the number of homologous amino acids in relation to the total number of amino acids. Thus, for example, homology of sequences shorter than an optimum can be determined using the number of amino acids in the shorter sequence.

Moreover, as genetic sequences can vary between different HSV strains, the natural scope of allelic variation is included within the scope of the invention. In this respect, the inventive polypeptide can be or comprise mutants (particularly point substitutions) of the exemplary sequences or other known HSV gD sequences or derivatives thereof. Typically, such mutations are conservative in nature, according to which positively-charged residues (H, K, and R) preferably are substituted with positively-charged residues; negatively-charged residues (D and E) preferably are substituted with negatively-charged residues; neutral polar residues (C, G, N, Q, S, T, and Y) preferably are substituted with neutral polar residues; and neutral non-polar residues (A, F, I, L, M, P, V, and W) preferably are substituted with neutral non-polar residues. In other embodiments, the inventive polypeptide can contains an insertion, deletion, or non-conservative substitution of at least 1 amino acid (e.g., from about 1 to about 5 or about 10 or more amino acids, such as up to about 20 or more amino acids or even an entire non-native domain) at the amino terminus, carboxyl terminus, and/or internally. Indeed, many functional mutants are indicated in Table 1 (employing Δ to indicate deletions of amino acids and AxxB to indicate substitutions, wherein A refers to the native residue, xx refers to the position of the native residue in the native gD sequence, and B refers to the substituted residue). Moreover, the inventive polypeptide also can include other domains, such as epitope tags and His tags, nuclear localization signals, antigenic domains or epitopes, etc. (e.g., the inventive polypeptide can be a fusion protein).

The inventive polypeptide can be synthesized by any desired method. For example, it can be made using standard direct peptide synthesizing techniques (e.g., as summarized in Bodanszky, Principles of Peptide Synthesis (Springer-Verlag, Heidelberg: 1984)), such as via solid-phase synthesis (see, e.g., Merrifield, J. Am. Chem. Soc., 85, 2149-54 (1963); Barany et al, Int. J. Peptide Protein Res., 30, 705-739 (1987); and U.S. Pat. No. 5,424,398). As polynucleotides encoding suitable proteins are known or can be deduced from the polypeptide sequences disclosed herein, the polypeptide can be produced by standard recombinant methods, if desired.

However, produced, and depending on the desired end use, the polypeptide can be formulated into a suitable composition, which can include other ingredients such as carriers, excipients, diluents, biologically-active compounds, etc., as desired. For example, to facilitate long-term storage, the polypeptide can be lyophilized or otherwise desiccated. Accordingly, the invention provides a composition including the inventive polypeptide in such form. Alternatively, a composition can include the polypeptide and a protein-stabilizing agent, such as an aqueous or organic solvent, a sugar (e.g., glucose, trahalose, etc.), or other suitable stabilizing agents, and the invention provides such a composition.

For use in vivo, the invention provides a pharmaceutical (including pharmacological) composition comprising the inventive polypeptide and a suitable diluent. The diluent can include one or more pharmaceutically- (including pharmacologically- and physiologically-) acceptable carriers. Pharmaceutical compositions for use in accordance with the present invention can be formulated in a conventional manner using one or more pharmaceutically- or physiologically-acceptable carriers comprising excipients, as well as optional auxiliaries that facilitate processing of the inventive polypeptide into preparations that can be used pharmaceutically. Proper formulation is dependent upon the route of administration chosen. Thus, for systemic injection, the inventive polypeptide can be formulated within aqueous solutions, preferably in physiologically-compatible buffers. For transmucosal administration, penetrants appropriate to the barrier to be permeated are used in the formulation. Such penetrants are generally known in the art. For oral administration, the inventive polypeptide can be combined with carriers suitable for inclusion into tablets, pills, dragees, capsules, liquids, gels, syrups, slurries, liposomes, suspensions and the like. For administration by inhalation, the inventive polypeptide is conveniently delivered in the form of an aerosol spray presentation from pressurized packs or a nebulizer, with the use of a suitable propellant. The inventive polypeptide can be formulated for parenteral administration by injection, e.g., by bolus injection or continuous infusion. Such compositions can take such forms as suspensions, solutions or emulsions in oily or aqueous vehicles, and can contain formulatory agents such as suspending, stabilizing and/or dispersing agents. For application to the skin, the inventive polypeptide can be formulated into a suitable gel, magma, cream, ointment, or other carrier. For application to the eyes, the inventive polypeptide can be formulated in aqueous solutions, preferably in physiologically compatible buffers. The inventive polypeptide also can be formulated into other pharmaceutical compositions such as those known in the art. In particular, for use on skin, mucosal tissues, or in conjunction with prophylactic agents, the composition can include commonly employed constituents such as antibiotic agents, antiviral agents, protein stabilizing agents, spermicidal agents, lubricants, etc. Moreover, for use in or as a vaccine, the composition can include vaccine adjutants such as are routinely used (e.g., Freund's adjuvant, aluminum hydroxide, and aluminum potassium sulfate, etc.).

A composition including the inventive polypeptide can be packaged to facilitate a desired end use in accordance with standard methods of packaging. Thus, for example, for internal use in vivo, the composition can be packaged within a suitable vial or a syringe, and the invention provides a syringe comprising a composition including the inventive polypeptide and/or a composition containing the polypeptide, such as are set forth herein. In other embodiments, the composition including the inventive polypeptide can further include, and be packaged with, a prophylactic device or barrier such as are commonly used to resist the passage of biological material between individuals (e.g., condoms, gloves, safety eyeglasses or goggles, vaginal inserts (such as diaphragms, sponges, and the like) or other suitable prophylactic devices or barriers). In such a preparation, the inventive polypeptide (typically within a composition as described above) can bathe or coat the device or barrier, and preferably it covers the entirely of the surface exposed either to the individual wearing the device or barrier or the environment. Indeed, even greater protection is achieved when the composition coats the entirety of all surfaces. By applying the inventive polypeptide (e.g., within a composition such as discussed above) to such a prophylactic device, the invention provides a method of increasing the likelihood that the device will resist HSV or HIV infection of an individual on which such a device has been properly disposed. While the inventive method need not provide failsafe protection, any increase in the likelihood that the device will resist the spread of such infectious agents can improve the safety of such devices.

In one embodiment, the inventive polypeptide is a ligand for cell surface proteins associated with HSV and/or HIV attachment and/or infection (e.g., HveC and/or HveA). Such polypeptides can attenuate or even block binding of live virus and, therefore, reduce the ability of live HSV or HIV to infect the cells. Accordingly, the invention provides a method of protecting a cell from infection with HSV or HIV. In accordance with this method, the inventive polypeptide (or, in other embodiments, an isolated wild-type gD polypeptide) is placed into contact with the surface of the cell under conditions sufficient for the polypeptide to associate with the surface of the cell so as to interfere with the ability of the cell to infectively interact with HSV or HIV. Subsequently, when a live virus (e.g., within a composition such as a biological solution such as blood, lymph, saliva, wound exudates, urine, semen, tears, etc. or an artificial solution containing the virus) contacts the cell, it is less likely to bind the cell as required for infection. For example, where the polypeptide is a ligand for HveA and/or HveC, it can bind such protein when present on the surface of the cell and block infection. Any interaction between the polypeptide and the cell that reduces the probability of subsequent viral infection is within the scope of the inventive method, regardless of which cell surface proteins are involved. In this regard, the cell need not be completely insulated from all possibility of viral infection; it is sufficient for the likelihood to be reduced. The degree to which the practice of the inventive method reduces the likelihood of infection correlates to the amount of protein exposed to the cell surface.

While the method of protecting a cell can be employed in vitro (e.g., as a research tool to investigate the mechanism of viral infectivity), it also can be employed in vivo (e.g., applied to protect populations of cells, tissues, organs, etc.). Indeed, the method can protect whole organisms from viral infection. In this mode, the invention provides a method of reducing the probability of HSV or HIV infection of an individual upon exposure to infectious HSV or HIV. Accordingly, the method can be employed to reduce the spread of HSV or HIV from an HSV⁺ or HIV⁺ individual to an HSV⁻ or HI⁻ individual during physical contact between the individuals. In this context, the HSV⁺or HIV⁺individual caries a strain of HSV or HIV that the HSV⁻ or HIV⁻ individual does not carry. In accordance with this method, the inventive polypeptide (or, in other embodiments, an isolated wild-type gD polypeptide), typically within a composition, such as described above, is applied to at least that portion of the surface (e.g., skin, open wounds, mucous tissue, buccal epithelium, ocular epithelium, oral epithelium, nasal epithelium, genital epithelium, anal epithelium, etc.) of at least one of the individuals that is in contact with (or is likely to come into contact with) the other individual prior to the physical contact between the individuals. The polypeptide can be applied topically or in conjunction with the application of a prophylactic device, or both, as desired. Desirably, the polypeptide is applied to the actual or likely contact surfaces, or even the entire or substantially entire surfaces, of both individuals, although this is not necessary to achieve enhanced protection in all cases. By virtue of the presence of the polypeptide on the surface of at least one individual, at least some fraction (and desirably all) of the viral cell-surface receptors is blocked from contacting the virus, at least in a manner sufficient to permit infection. The degree to which such receptors are blocked, and the number that are blocked, depends on the concentration of the polypeptide on the surface, and whether the surfaces of one or both individuals are treated. However, as discussed above, any degree of cell blocking also reduces the likelihood that the HSV⁻ or HIV⁻ individual will become infected. While the method can be applied to humans, it also can be used on non-human mammals. Indeed, application to such animals (preferably primates) can be used to test the efficacy of the inventive method.

In another embodiment, the inventive polypeptide can be immunogenic and able to potentiate an immune response against HSV. Accordingly, the invention provides a method of vaccinating an individual (e.g., a human patient) using the inventive polypeptide (or, in other embodiments, an isolated wild-type gD polypeptide). In accordance with the method, an amount of the polypeptide is introducing into the individual under conditions sufficient for the individual to develop an immune response to HSV. Typically, the polypeptide is introduced into the patient after formulating it into a composition, such as discussed above, preferably a pharmaceutically acceptable composition. Such a composition can be introduced into the individual in accordance with accepted means of vaccination, e.g., by subdermal, subcutaneous, or intramuscular injection, or by other desired methods. However, introduced into the patient, a sufficient quantity of the polypeptide should be introduced into the individual so as to potentiate an immune response. In this context, immune response can be assessed using any standard measure of the degree to which an inoculee's immune system is primed against subsequent exposure to HSV (especially to gD protein). Typically, a dose should deliver about 0.1 μg/kg individual weight to about 10 μg/kg individual weight, although the optimum dose can vary from this guideline, as desired. Moreover, the method can employ repeated or “booster” inoculations, as appropriate.

### EXAMPLES

While one of skill in the art is fully able to practice the instant invention upon reading the foregoing detailed description, the following examples will help elucidate some of its features. In particular, they reveal that the inventive polypeptide is a ligand for cell surface proteins associated with HSV binding and internalization and that exposure of cells to the inventive polypeptide can block subsequent HSV infection. As these examples are presented for purely illustrative purposes, they should not be used to construe the scope of the invention in a limited manner, but rather they should be seen as expanding upon the foregoing description of the invention as a whole.

The procedures employed in these examples, such as gene cloning, protein synthesis, manipulation of viral genomes, ELISA, and cell culture and assay are familiar to those of ordinary skill in this art (see, e.g., Sambrook et al, Molecular Cloning: A Laboratory Manual, 2d edition, Cold Spring Harbor Press (1989); see also Watson et al., Recombinant DNA, Chapter 12, 2d edition, Scientific American Books (1992)). As such, and in the interest of brevity, experimental protocols are not recited in detail.

### Example 1

This example demonstrates that the inventive polypeptide is a ligand for cell surface proteins associated with HSV binding and internalization.

Polypeptides A1 (SEQ ID NO:1) and A2 (SEQ ID NO:25), corresponding to residues 7-27 and residues 1-33 of the gD protein, respectively, were synthesized according to standard methods of protein synthesis. ELISA plates were coated with 400 ng/well HveA (200t) or HveC (346t), blocked, and incubated with various concentrations (between 1 μM and 1000 μM) of the A1 or A2 polypeptides. Bound peptides were detected with antiserum R11, followed by peroxidase-conjugated secondary antibody and substrate.

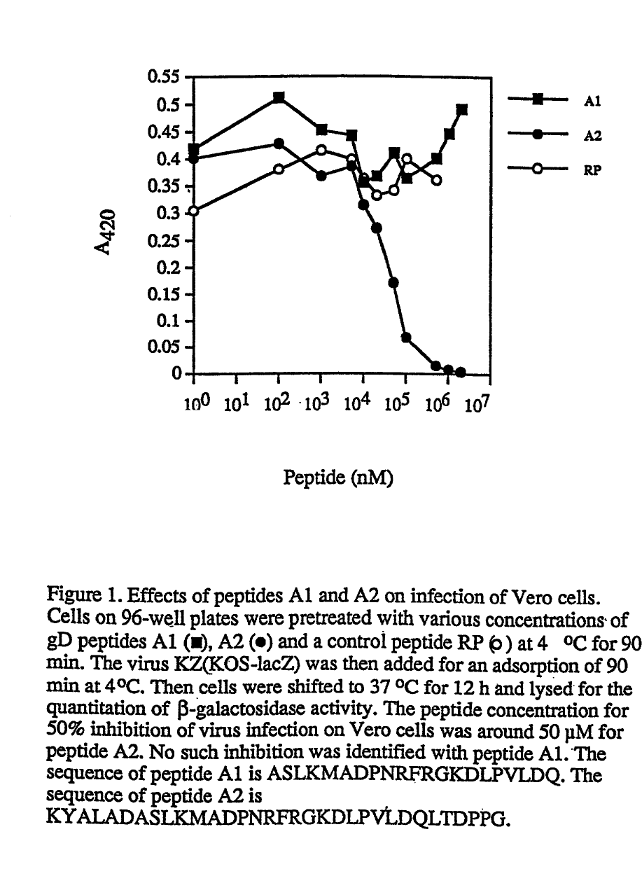

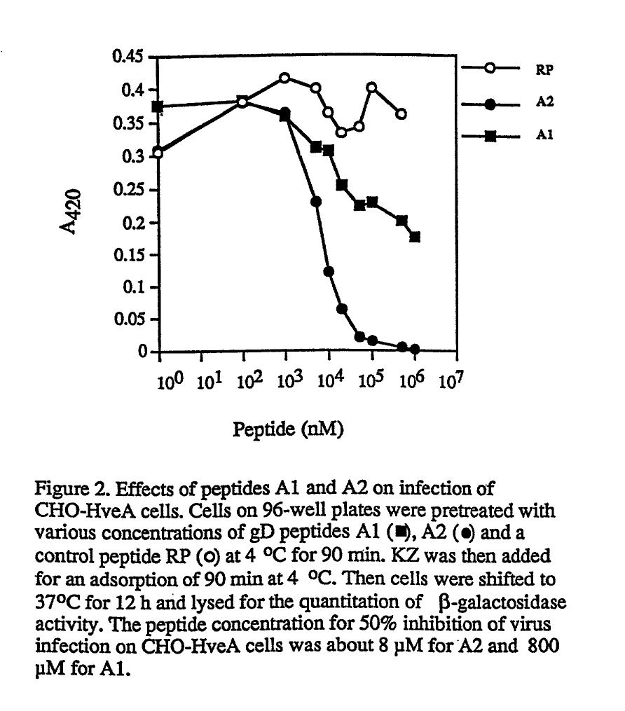

The experiment was repeated several times, and the data for duplicate wells were averaged to assess experimental results. The data reveal that the A2 polypeptide binds to recombinant HveC and HveA proteins, whereas the shorter A1 polypeptide does not. The results of these experiments, indicating the varying binding affinities of gD peptides to HveA or HveC, are indicated in FIGS. 1 and 2.

### Example 2

This example demonstrates that exposure of cells to the inventive polypeptide can block subsequent HSV infection.

The cells employed in these experiments were well known Vero cells, as well as Chinese hamster ovary (CHO) cells engineered to express either recombinant HveA (i.e., “CHO-HveA cells”) or HveC (i.e., “CHO-HveC cells”).

The virus employed in these experiments (KZΔUs3-8) is a gD complemented HSV-1 KOS strain mutant having the βgalactosidase gene.

After a few days incubation, the cells were pretreated with various concentrations polypeptides A1, A2 or a control peptide at 4° C. for 90 minutes. The KZΔUs3-8 virus then was added for an adsorption of 90 minutes at 4° C. The cells were shifted to 37° C. for 12 hours and lysed for the quantitation of β-galactosidase activity.

The peptide concentration for 50% inhibition of virus infection on Vero cells was around 50 μM for peptide A2, and no such inhibition was identified with either peptide A1 or RP. The peptide concentration for 50% inhibition of virus infection on CHO-HveA cells was about 8 μM for A2 and 800 μM for A1. The peptide concentration for 50% inhibition of virus infection on CHO-HveC cells was about 30 μM for A2, and no inhibition was identified with peptide A1 or RP.

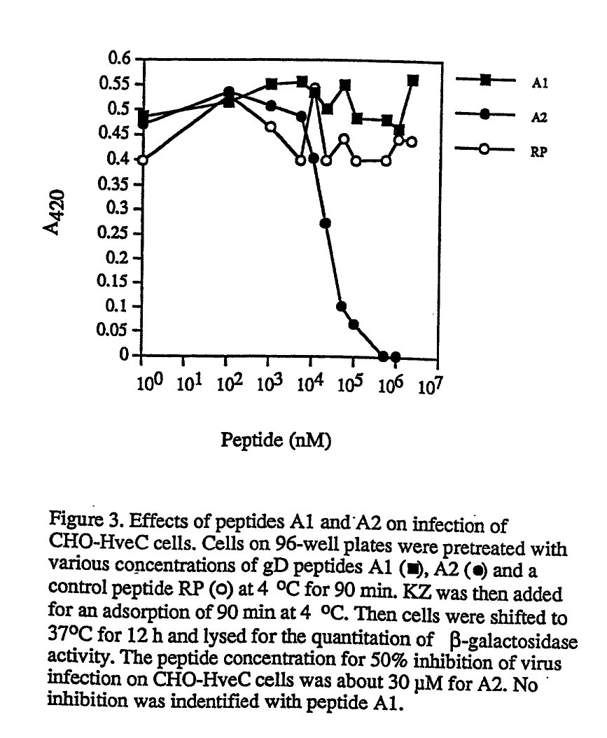

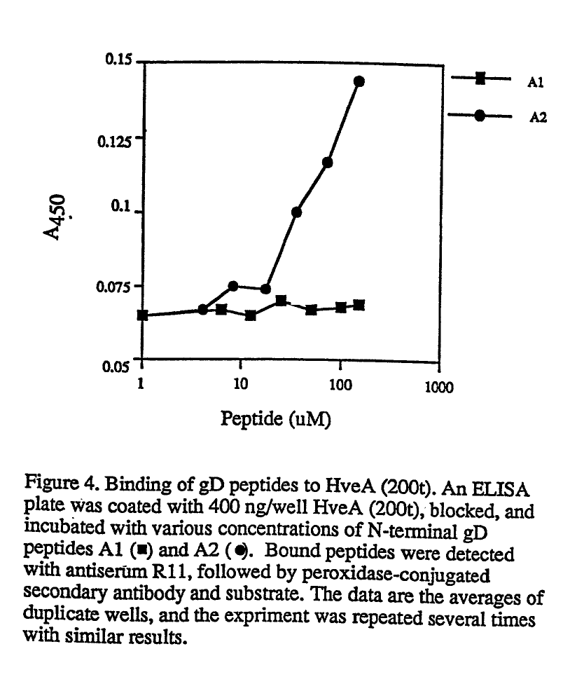

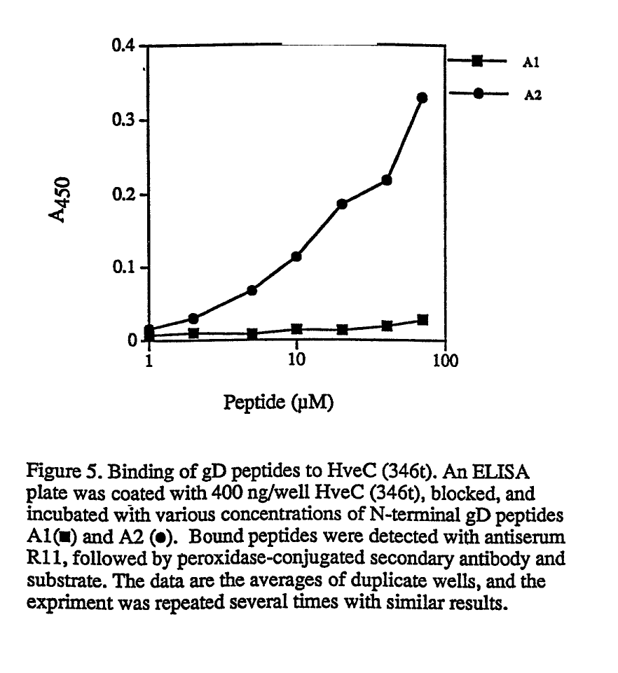

These results, depicted in FIGS. 3-5, reveal that the A2 polypeptide blocked infection of Vero cells as well as the engineered CHO-HveA and CHO-HveC cells, whereas the shorter A1 polypeptide did not effectively block entry to any of the tested cells.

### Example 3

This example demonstrates the properties of several mutant gD proteins having mutations in the amino-terminal region. The results of the experiments set forth herein are presented in Table 1.

Vero cells were obtained from the ATCC. VD60 is a gD-complementing cell line. Vero and VD60 were grown in Dulbecco's modified Eagle's medium (DMEM; Gibco) supplemented with 10% fetal bovine serum (FBS). CHO-K, CHO-HveA and CHO-HveC cells were grown in F-12K medium (GIBCO) supplemented with 10% FBS. All cell lines were maintained at 37° C.

All mutants and recombinant virus strains used in this Example were derivatives of HSV-1 strain KOS. KZ is a LacZ+ virus generated by insertion of an HCMV IE promoter-driven lacZ gene into the thymidine kinase (tk) locus of KOS. KΔUS3-8Z, a gD-null LacZ+ virus, has been described previously (Anderson et al., J Virol., 74, 2481-87 (2000)

The HSV-1 SacI fragment, containing the gD promoter and gD open reading frame, was cloned into plasmid pSP72 (PROMEGA). The resulting construct was named pSP72-gD. All gD mutant genes were derivatives of pSP72-gD.

gD deletion mutants were constructed using the Gene Editor in vitro site-directed mutagenesis kit (PROMEGA). Briefly, the kit's selection oligonucleotide and a mutagenic primer specifying the deletion (Δ, del) were annealed to the appropriate gD template, such as pSP72-gD. Following DNA synthesis and mutant-strand ligation, mutants were selected for resistance to both ampicillin and the Gene Editor antibiotic selection mix included in the kit. Mutants were verified by DNA sequencing. Negative-control plasmid pgD−, containing a 4-nucleotide substitution of codons 5-28 causing a frame-shift while creating a unique PacI site, was generated on the pSP72-gD template using mutant primer. No gD product was detected upon pgD⁻ expression. Deletion mutants obtained using pgD⁻ as template were pgDΔ6-27, which also copied a portion of the PacI site specifying an amino-acid change at position 5 (A5I), and intermediate plasmid pgDΔ7-39 where the deletion created a unique EcoRV site. Intermediate deletion constructs pgDΔ31-39 and pgDΔ47-54 were generated on wild-type gD template pSP72-gD using mutagenic primers that created a unique EcoRV site at the deletion boundary in both cases. pgDΔ31-39/D26G was subsequently derived from pgDΔ31-39 by GENE EDITOR mutagenesis changing 2 basepairs at codons 25 and 26 to generate an AvrII site which resulted in the D26G substitution. GENE EDITOR mutagenesis was also used to generate deletion mutant pgDΔ2-5 on pSP72-gD template. Additional deletion mutants (pgDΔ6-9, pgDΔ10-16, pgDΔ17-21, pgDΔ22-24, pgDΔ6-24, and pgDΔ6-24:GSK) were derived from pgD⁻ by PacI digestion and insertion of appropriate linkers with 3′ AT overhangs at both ends. Each insertion regenerated the A5I mutant codon at the PacI cleavage site. In pgDΔ6-24:GSK, the linker replaced codons 6-27 with a sequence encoding the unrelated tripeptide GSK which introduced a unique BamHI site.

For amino acid substitution mutations, each selected codon was replaced by a codon library of sequence 5′-NNY-3′ (N, any nucleotide; Y, pyrimidine). Briefly, degenerate upper- and lower-strand oligonucleotides containing, respectively, 5′-NNY-3′ and 5′-RNN-3′ (R, purine) at the selected codon position between complementary sequences were annealed by heating at 95° C. for 5 min. and slow cooling to room temperature. Where suitable, oligonucleotide pairs were designed to leave sticky ends matching the ends of restriction enzyme-digested gD plasmid DNA. Following ligation at 16° C. for 12-18 h and bacterial transformation, plasmid DNAs were isolated from multiple colonies and individually characterized for transient complementation of the entry deficient gD− virus KΔUS3-8Z. Based on their complementation phenotypes, selected mutants were further characterized in receptor-binding assays and by DNA sequencing. NNY libraries for positions 6, 7, 8, and 9 were constructed by ligation of annealed oligonucleotides with 3′ AT overhangs to PacI-linearized pgD−. In each case, insertions in the sense orientation regenerated the A5I mutant codon of pgD−. For the construction of NNY libraries at positions 25, 26, and 27, an intermediate plasmid was derived from pgDΔ7-39. A blunt-ended linker with internal mutations generating recognition sites for EcoRV and BamHI straddling a frameshifting net deletion of 17 basepairs (R21-30EB and R21-30EB/C) was inserted at the unique EcoRV site of pgDΔ7-39, eliminating this site and creating pgDR21-30EB. Libraries were subsequently constructed by introduction of the respective NNY linkers (annealed pairs of NNY/RNN oligonucleotides), featuring one blunt end and a BamHI-compatible overhang, between the unique EcoRV and BamHI sites of pgDR21-30EB. As parental construct for the generation of NNY libraries at positions 28-32, plasmid pgD:26G33H was produced by insertion of a linker between the AvrII and EcoRV sites of pgDΔ31-39/D26G. The linker recreated the upstream AvrII site and the associated D26G mutation, but not the downstream EcoRV site, and introduced base changes at codons 33 and 34 creating a unique PmlI site and an amino-acid change (G33H). NNY linkers with one AvrII-compatible and one blunt end were inserted between the AvrII and PmlI sites of pgD:26G33H, in the process restoring codons 26 and 33 to wild-type. NNY libraries at positions 35 and 36 were generated by cloning of annealed pairs of NNY/RNN oligonucleotides into the EcoRV site of pgDΔ31-39. The vector used for library construction at positions 40, 41, and 44 was a multi-step derivative of pgDΔ31-39. First, pgD:H39V was created by insertion of a linker restoring positions 31-38 followed by a mutant codon 39 (H39V) to generate a unique SnaBI site. pgDΔ40-44SB containing a deletion of codons 40-44 and a silent mutation in codon 46 creating a unique BamHI site was subsequently derived by replacement of the SnaBI-BssHII fragment of pgD:H39V (codons 39-64) with a synthetic fragment restoring the SnaBI and BssHII sites. Finally, the unique SnaBI and BamHI sites of pgDΔ40-44SB were used for the construction of NNY libraries at positions 40, 41, and 44 using annealed oligonucleotides with one blunt end and a BamHI-compatible overhang. NNY libraries at positions 49-52 were constructed by insertion of blunt-ended NNY/RNN linkers into the unique EcoRV site of pgDΔ47-54.

In a first transient complementation assay, several cell lines were employed. VD60 cells express wild-type gD endogenously which complements the deleted gD gene of KΔU_{S}3-8Z for plaque formation, but only if the virus can initially infect using the gD product of the transfected gene. Thus, plaque formation on VD60 cells indicates complementation of gD's attachment/entry function by the transfected gene. CHO cells lack gD receptors and are resistant to HSV infection, but CHO cells transduced with HveA or HveC expression plasmids (CHO-HveA and CHO-HveC cells, respectively) are susceptible. KΔU_{S}3-8Z misses the complete gD gene (U_{S}6) due to a large deletion extending from U_{S}3 to U_{S}8 and therefore offers no target for homologous recombination with transfected gD genes or the stable gD gene of VD60 cells. Hence, although the virus will incorporate the product of the transfected gene in its envelope potentially enabling it to infect receptor-bearing cells, it is not genotypically altered and will therefore be limited to one round of infection on gD-negative cells like CHO-HveA and CHO-HveC cells. Since the progeny virus lacks gD, plaques will not form on these cells and virus entry was therefore determined by measurement of lacZ reporter gene expression.

To conduct the assay, Vero cells were transfected with LIPOFECTAMINE-PLUS (GIBCO) for 4 h at 37° C., the cell monolayers washed and incubated with DMEM/10% fetal bovine serum (FBS) for 16 h at 37° C., and the transfected cells infected with KΔU_{S}3-8Z at an MOI of 3 for 2 h at 37° C. After removal of the medium and inactivation of residual extracellular virus by incubation of the monolayer with 0.1M glycine (pH 3.0) for 1 min at room temperature, fresh medium was added and the cells incubated at 37° C. for 48 hours. The medium was subsequently removed and temporarily stored on ice while the cells were being lysed by freeze-thawing and sonication. Cell debris was pelleted by low-speed centrifugation and the supernatant combined with the previously stored medium. Virus titers were determined on gD-complementing VD60 cells. Complementing activity was determined by infection of CHO-HveA, CHO-HveC, and control CHO-K cells. Infected cells were lysed in a buffer containing 1% NP-40, 1 mM MgCl₂, 50 mM β-mercaptoethanol, and 4 mg/ml β-galatosidase substrate O-nitrophenyl β-D-galactopyranoside (ONPG, Sigma) in a total volume of 50 μl. The enzyme-substrate reaction was carried out at 37° C. and stopped by addition of an equal volume of 1M Na₂CO₃ after color development. β-galactosidase activity was measured by reading the absorbance at 420 nm. One hundred percent complementation was defined as the difference between the A₄₂₀ values obtained for the wild-type (pSP72-gD) and negative control (pgD⁻) gD plasmids. Relative complementation efficiencies were calculated as 100%×[A₄₂₀(mutant)−A₄₂₀(gD⁻)]/[A₄₂₀(wild type)−A₄₂₀(gD⁻)].

In another procedure, 293T cells were transfected with gD plasmid and infected with KΔU_{S}3-8Z as described above. Following incubation for 16 h at 37° C., the cells were washed with phosphate-buffered saline (PBS) and lysed in 1% NP-40 lysis buffer. The supernatant was collected by centrifugation and the protein concentration of each sample determined by Bio-Rad protein assay. Identical amounts of protein were electrophoresed on SDS-polyacrylamide gels and the proteins electroblotted to nitrocellulose membranes in a 5% solution of dry milk in PBST (0.1% Tween-20 in PBS, pH 7.0) for 1 h at room temperature. The membranes were washed, incubated with a 1:10,000 dilution of R7 rabbit polyclonal anti-gD antiserum in 5% milk/PBST for 16 h at 4° C., washed again, and incubated with 1:20,000-diluted horseradish peroxidase-conjugated goat anti-rabbit antibody (Sigma) for 1 h at room temperature. After several more washes, the membranes were developed using an Amersham ECL kit.

Receptor-binding assays also were conducted, in which soluble gD receptors [HveA(200t), HveC(346t)] were purified. 250 ng HveA(200t) or 200 ng HveC(346t) in PBS (pH 9.2) were bound to each well of 96-well enzyme-linked immunosorbent assay (ELISA) plates overnight at 4° C. The wells were subsequently washed three times with PBST and incubated for 1 h at 37° C. in BLOCKING AND SAMPLE BUFFER (PROMEGA). After three more washes, the wells were incubated with lysates of gD plasmid-transfected, KΔU_{S}3-8Z virus-infected Vero cells in BLOCKING AND SAMPLE BUFFER for 16 h at 4° C. Following an additional five washes with PBST, the wells were incubated for 1 h with R7 anti-gD antiserum diluted 1:1,000 in Blocking and Sample Buffer, washed another five times, and incubated with horseradish peroxidase-conjugated goat anti-rabbit antibody (SIGMA) diluted 1:40,000 in Blocking and Sample Buffer. The plates were finally washed again and TMB substrate solution (SIGMA) added. The enzyme reaction was stopped by addition of an equal volume of 2N H₂SO₄ and the enzyme activity measured by reading the absorbance at 450 nm.

To confirm the involvement of the N-terminal region of gD in HveA-, but not HveC-dependent HSV entry, a gD deletion mutant missing amino acids 6-24 (gDΔ6-24) compared to wild-type (wt) gD and a frame-shifted mutant gene (gD⁻) in which codons 5-28 were replaced by a 4-nucleotide sequence creating a PacI site. After transfection of Vero cells, the gDΔ6-24 mutant protein was detected by Western blotting and immunofluorescent staining demonstrating cell surface expression. Receptor binding was assessed by ELISA using lysates from transfected cells and baculovirus-produced, C-terminally truncated recombinant HveA or HveC protein (Krummenacher et al., J. Virol. 72, 7064-74 (1998); Willis et al., J. Virol., 72, 5937-47 (1998)). The results demonstrated capture of gDΔ6-24 by immobilized HveC, but not HveA protein, as determined relative to similarly tested wild type gD and gD⁻ (Table 1). Complementation assays showed that gDΔ6-24 did not enable entry of the gD-deficient virus KΔU_{S}3-8Z into CHO-HveA cells, consistent with the inability of the mutant protein to interact with recombinant HveA, while entry into VD60 and CHO-HveC cells was restored (Table 1).

The gDΔ6-24 gene, like other derivatives of the gD⁻ gene presented herein, had an isoleucine codon at position 5 instead of the wild-type alanine codon (A5I mutation) reflecting some of the changes that created the PacI site of gD⁻. Using a rescue plasmid with just the A5I substitution (gD⁻R), this mutation was observed to have essentially no effect on the receptor-binding and complementation properties of gD (Table 1), indicating that the defects ascribed to deletions or substitutions in gD⁻-derived constructs were not caused by the A5I mutation.

To explore the complexity of the N-terminal region involved in HveA binding, smaller deletions were tested (residues 2-5, 6-9, 10-16, 17-21, and 21-24). Each mutant protein was expressed and detected on the cell surface similarly to wild-type gD. None could complement KΔU_{S}3-8Z for entry into CHO-HveA cells, although all were comparable to wild-type gD in directing virus into VD60 and CHO-HveC cells (Table 1). In addition, the complemented viruses were fusion-competent, at least when initiated by HveC binding.

To further explore the sensitivity of HveA-dependent entry to changes in the N-terminal sequence of gD, codons 6, 7, 8, and 9 were separately replaced by the sequence NNY to generate position-specific mutant libraries from which randomly selected mutants were tested for complementation. The results showed that the majority of mutants at each position were complementation-positive on VD60 and CHO-HveC cells but negative on CHO-HveA cells, the same phenotype as the deletion mutants. Several of these mutants were sequenced, and binding experiments with purified recombinant HveA and HveC demonstrated that gD binding to HveA was disrupted (Table 1). Among the substitutions that caused this defective HveA binding were subtle changes such as A7L, S8L, and L9A; however, two non-conservative mutations (A7H, S8A) also left residual complementing activity on HveA cells (40-80% of wt gD, Table 1). A charge-altering mutation at position 1 produced no phenotypic change in complementing activity compared to wild-type gD (Table 1).

Whereas gDΔ6-24 retained full complementing activity on CHO-HveC cells, additional deletion (gDΔ6-27) or mutation (gDΔ6-24:GSK) of the next three amino acids resulted in substantially reduced entry into CHO-HveC and VD60 cells although HveC binding was comparable to wild-type (Table 1). No binding to HveA or entry into CHO-HveA cells was observed. Thus, one or more residues at positions 25-27 contribute to HveC-dependent entry. To determine which of the positions 25-27 were important for HveC-dependent entry, the three codons were individually randomized and sets of undefined mutants at each position screened in the complementation assay. All 27 mutants at position 25 were complementation-deficient on CHO-HveA (A⁻), CHO-HveC (C⁻), and VD60 cells (less than 30% of wild-type activity), indicating that position 25 plays a role in entry via both HveA and HveC. Four of the random mutants at position 25 were sequenced (L25D, L25H, L251, and L25T) and the corresponding proteins detected by immunoblotting as well as cell-surface immunostaining, indicating adequate expression and proper processing. Like gDΔ6-27 and gDΔ6-24:GSK, these mutants were capable of HveC but not HveA binding (Table 1). Position 25 therefore is involved in mediating both binding of gD to HveA and fusion initiated by binding to HveC.

Multiple individual positions C-terminal to residue 25 were mutated to the degenerate NNY sequence and each of the resulting position-specific mutant libraries sampled in the complementation assays. Several representative as well as unusual isolates at each position were examined for expression, sequenced, and the proteins tested by ELISA for receptor binding. As before, mutants were scored as complementation-positive if they demonstrated greater than 30% activity compared to wild-type gD on CHO-HveA cells (A⁺) or CHO-HveC cells (C⁺). Some wild-type background was expected at almost all positions since the mutagenic NNY sequence could regenerate the wild-type residue at a frequency of 6% in all cases except at positions 27, 41, and 49 (0%). Complete data for individual mutants are shown in Table 1. Mutations at positions 49-52 had no effect on complementation; whereas mutants with diminished complementing activity on CHO-HveC cells were observed at all other analyzed positions C-terminal to residue 25 (i.e., through residue 44 minimally and 48 maximally).

Within the region bounded by residues 25 and 48, mutants were isolated at positions 28 (L28P and L28G), 29 (T29G and T29Y), 31(P31G), and 32 (P3). Since these mutants were unimpaired for interaction with HveC, their defective binding to HveA could not be ascribed to reduced expression or faulty processing, indicating instead that the affected residues are components of or contribute to the presentation of the HveA binding surface.

Several mutations resulted in proteins that could not bind to HveC or to HveA and thus were entry-deficient. Examples were found at positions 36 (R36A, R361, and R36D), 40 (140N and 140A), and 41 (Q41P). Cell-surface expression was similar for all these mutants and apparently normal. None of the screened mutants at positions 36 and 40 showed complementing activity on the two indicator cell lines, and no binding-competent mutants were identified. In contrast, the mutant identified at position 41 was complementation defective. These observations suggest that positions 36 and 40 are determinants of both the HveC and HveA ligands of gD. Although the exceptional mutation at position 41 (glutamine to proline) may affect folding, the complementation competence of the majority of mutants at this position argued that position 41 is not a critical component of the two ligands.

Several mutations resulted in proteins that bound to both receptors, but could mediate infection only of HveC-positive cells. Two of the five mutants characterized at position 28 were of this type (L28C and L28S), with a third showing reduced complementing activity on CHO-HveC cells (L28F). A specific mutant generated at position 33 (G33L) also showed similar properties.

Several mutations resulted in proteins that were complementation deficient but capable of HveC binding. In addition to the four mutants at position 25 presented above, all four randomly characterized mutants at position 27 (Q27H, Q27S, Q27T, and Q27G) were of this type, although the Q27P rid1 mutant can mediate entry via HveC. These mutants also included the three mutants at position 30 (D301, D30V, and D30H) although all three exhibited reduced binding to HveC.

Other mutations resulted in proteins that were competent for binding to both receptors but negative in complementation tests. Among this group of mutations are the minority mutant L44G and three characterized mutants at position 26 (D26H, D26S, and D26V).

### INCORPORATION BY REFERENCE

All sources (e.g., inventor's certificates, patent applications, patents, printed publications, repository accessions or records, utility models, world-wide web pages, and the like) referred to or cited anywhere in this document or in any drawing, Sequence Listing, or Statement filed concurrently herewith are hereby incorporated into and made part of this specification by such reference thereto.

### INTERPRETATION GUIDELINES

The foregoing detailed description sets forth “preferred embodiments” of this invention, including the best mode known to the inventors for carrying it out. Of course, upon reading the foregoing description, variations of those preferred embodiments will become obvious to those of ordinary skill in the art. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law.

As used herein, singular indicators (e.g., “a” or “one”) include the plural, unless otherwise indicated. The term “consisting essentially of” indicates that unlisted ingredients or steps that do not materially affect the basic and novel properties of the invention can be employed in addition to the specifically recited ingredients or steps. In contrast, the terms “comprising” or “having” indicate that any ingredients or steps can be present in addition to those recited. The term “consisting of” indicates that only the recited ingredients or steps are present, but does not foreclose the possibility that equivalents of the ingredients or steps can substitute for those specifically recited.

TABLE I

| | % Complementation^{a} | | | | Binding | |
|---|---|---|---|---|---|---|
| Mutant | | CHO- | CHO- | Surface | ability^{c} | |
| name | VD60 | HveA | HveC | Expression^{b} | HveA | HveC |
| wt | 100 | 100 | 100 | + | + | + |
| gD⁻ | 0 | 0 | 0 | − | − | − |
| gD⁻_{R} | 100 | 85 | 100 | + | n.d.^{e} | n.d. |
| K1D | 100 | 100 | 100 | n.d. | n.d. | n.d. |
| D6A | 100 | 0 | 99 | + | − | + |
| D6L | 100 | 0 | 98 | + | − | + |
| A7H | 100 | 80 | 100 | n.d. | n.d. | n.d. |
| A7L | 100 | 0 | 100 | + | − | + |
| A7R | 100 | 0 | 98 | + | − | + |
| S8L | 100 | 0 | 99 | + | − | + |
| S8A | 100 | 40 | 100 | n.d. | n.d. | n.d. |
| L9A | 100 | 0 | 100 | + | − | + |
| L9R | 100 | 0 | 97 | + | − | + |
| L9P | 100 | 0 | 100 | n.d. | n.d. | n.d. |
| Δ2-5 | 100 | 0 | 95 | + | − | + |
| Δ6-9 | 100 | 0 | 100 | + | n.d. | n.d. |
| Δ10-16 | 100 | 0 | 100 | + | n.d. | n.d. |
| Δ17-21 | 100 | 0 | 100 | + | n.d. | n.d. |
| Δ21-24 | 100 | 0 | 100 | + | n.d. | n.d. |
| Δ6-24 | 100 | 0 | 100 | + | − | + |
| Δ6-27 | 12 | 0 | 31 | + | − | + |
| Δ6-24:GSK | 6 | 0 | 19 | + | − | + |
| L25D | 10 | 0 | 14 | + | − | + |
| L25H | 14 | 0 | 12 | + | − | + |
| L25I | 8 | 0 | 5 | + | − | + |
| L25T | 14 | 0 | 3 | + | − | + |
| D26H | 17 | 10 | 11 | + | + | + |
| D26S | 13 | 20 | 7 | + | + | + |
| D26V | 14 | 0 | 26 | + | + | + |
| Q27G | 0 | 0 | 0 | + | − | + |

| | % Complementation^{a} | | | | Binding | |
|---|---|---|---|---|---|---|
| Mutant | | CHO- | CHO- | Surface | ability^{c} | |
| names | VD60 | HveA | HveC | Expression^{b} | HveA | HveC |
| Q27H | 0 | 0 | 14 | + | − | + |
| Q27S | 0 | 0 | 9 | + | − | + |
| Q27T | 0 | 0 | 0 | + | − | + |
| Q27P(rid1) | 70 | 0 | 83 | + | − | + |
| L28C | 80 | 0 | 100 | + | + | + |
| L28P | 33 | 0 | 69 | + | − | + |
| L28F | 8 | 0 | 41 | + | + | + |
| L28G | 70 | 0 | 100 | + | − | + |
| L28S | 54 | 0 | 83 | + | + | + |
| T29G | n.d. | 0 | 100 | + | − | + |
| T29Y | n.d. | 0 | 100 | + | − | + |
| D30I | 0 | 0 | 4 | + | − | +/− |
| D30V | 0 | 0 | 8 | + | − | +/− |
| D30H | 0 | 0 | 6 | + | − | +/− |
| P31G | n.d. | 0 | 100 | + | − | + |
| P32H | n.d. | 6 | 100 | + | − | + |
| G33L | 46 | 9 | 79 | + | + | + |
| R36A | 0 | 0 | 0 | + | − | − |
| R36I | 0 | 0 | 0 | + | − | − |
| R36D | 0 | 0 | 0 | + | − | − |
| H39V | n.d. | 75 | 66 | n.d. | n.d. | n.d. |
| H39Q | 80 | 50 | 100 | n.d. | n.d. | n.d. |
| I40N | 0 | 0 | 0 | + | − | − |
| I40A | 0 | 0 | 0 | + | − | − |
| Q41P | 0 | 0 | 18 | + | − | − |
| L44P | n.d. | 53 | 71 | n.d. | n.d. | n.d. |
| L44C | n.d. | 69 | 83 | n.d. | n.d. | n.d. |
| L44G | 0 | 0 | 8 | + | + | + |
| AII 49-52^{d} | 100 | 100 | 100 | n.d. | n.d. | n.d. |

what is claimed is:

1. A polypeptide having an amino acid sequence consisting essentially of a sequence of at least ten contiguous amino acids from among the 55 amino-terminal amino acids of a wild-type HSV gD protein, wherein the polypeptide varies from a wild-type HSV gD protein amino acid sequence at least one residue, and wherein the polypeptide is a ligand for HveA or HveC.

2. The polypeptide of claim 1, which binds HveA.

3. The polypeptide of claim 1, which binds HveC.

4. The polypeptide of claim 1, having an amino acid sequence consisting essentially of one of SEQ ID NOs:25-72 or conservative substitutions thereof.

5. The polypeptide of claim 1, comprising a contiguous amino acid sequence consisting essentially of one of SEQ ID NOs:73-75 or conservative substitutions thereof.

6. The polypeptide of claim 1, containing at least one conservative amino acid substitution.

7. The polypeptide of claim 1, having an amino acid sequence consisting of one of SEQ ID NOs:25-72.

8. A polypeptide having an amino acid sequence consisting essentially of a sequence of at least ten contiguous amino acids from among the 55 amino-terminal amino acids of a wild-type HSV gD protein, wherein the polypeptide varies from a wild-type HSV gD protein amino acid sequence at least one residue, and wherein the polypeptide precipitates an immonological response protective against HSV.

9. The polypeptide of claim 8, having an amino acid sequence consisting essentially of one of SEQ ID NOs:25-72 or conservative substitutions thereof.

10. The polypeptide of claim 8, comprising a contiguous amino acid sequence consisting essentially of one of SEQ ID NOs:73-75 or conservative substitutions thereof.

11. The polypeptide of claim of claim 8, containing at least one conservative amino acid substitution.

12. The polypeptide of claim 8, having an amino acid sequence consisting of one of SEQ ID NOs:25-72.

13. A composition comprising the polypeptide of claim 1 in lyophilized or desiccated form.

14. A composition comprising the polypeptide of claim 1 and a protein-stabilizing agent.

15. A composition comprising the polypeptide of claim 1 and a physiologically acceptable carrier.

16. A composition comprising the polypeptide of claim 8 in lyophilized or desiccated form.

17. A composition comprising the polypeptide of claim 8 and a protein-stabilizing agent.

18. A composition comprising the polypeptide of claim 8 and a physiologically acceptable carrier.

19. A method of protecting a cell from infection with HSV or HIV, the method comprising contacting the surface of a cell with a polypeptide of claim 1 under conditions sufficient for the polypeptide to associate with the surface of the cell.

20. The method of claim 19, wherein the polypeptide is a ligand for HveA and the cell has an HveA protein on its surface.

21. The method of claim 19, wherein the polypeptide is a ligand for HveC and the cell has an HveC protein on its surface.

22. The method of claim 19, wherein the cell is in vivo.

23. A method of reducing the probability of HSV or HIV infection from an HSV⁺ or HIV⁺ individual to an HSV⁻ or HIV⁻ individual during physical contact between the individuals, the method comprising applying the composition of 15 to the contact surface of at least one of the individuals prior to the physical contact between the individuals, wherein the HSV⁺ or HIV⁺ individual caries a strain of HSV or HIV which the HSV⁻ or HIV⁻ individual does not carry.

24. The method of claim 23, wherein the surface is selected from the group of surfaces consisting of skin, open wounds, mucous tissue, ocular epithelium, nasal epithelium, genital epithelium, and anal epithelium.

25. The method of claim 23, wherein the composition is applied to the surface topically.

26. The method of claim 23, wherein the composition is applied to the surface in conjunction with the application of a prophylactic device.

27. A method of increasing the likelihood that a prophylactic device will resist HSV infection to an individual, the method comprising applying the protein of claim 1 to the prophylactic device.

28. The method of claim 27, wherein the polypeptide is within a composition comprising a carrier and one or more agents selected from the group of agents consisting of antibiotic agents, antiviral agents, protein stabilizing agents, spermicidal agents, and lubricants.

29. A composition for resisting the transmission of HSV comprising a prophylactic device and the polypeptide of claim 1.

30. The composition of claim 29, further comprising a carrier and one or more agents selected from the group of agents consisting of antibiotic agents, antiviral agents, protein stabilizing agents, spermicidal agents, and lubricants.

31. A method of vaccinating an individual comprising introducing the composition of claim 18 into an individual under conditions sufficient for the individual to develop an immune response to HSV.

32. The method of claim 31, wherein the composition is introduced into the individual by subdermal, subcutaneous, or intramuscular injection.

33. The method of claim 31, wherein a sufficient quantity of the composition is administered to the individual so as to deliver a polypeptide dose to the individual of from about 0.1 μg/kg individual weight to about 10 μg/kg individual weight.

34. The method of claim 31, wherein the individual is human.
